SEQUENCE LISTING (11 sequences)

SEQ ID NO: 1 - DNA, 746 bp, Homo sapiens
  CDS (56)...(442)
ccagcaggaggcacaggaaaactgcaagccgctctgttcctgggcctcggaagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctctttgcccttacccctaaataaagcaagcagttcttga

SEQ ID NO: 2 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 3 - protein, 106 aa, Homo sapiens
QEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 4 - DNA, 390 bp, Homo sapiens
  CDS (1)...(390)
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatc

SEQ ID NO: 5 - protein, 130 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 6 - protein, 106 aa, Homo sapiens
ASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 7 - DNA, 5605 bp, Homo sapiens
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggaggtgagttgctaagttgtgcagatgacagtgtcttctaggccagcagcttgggtctgattcttaagagttcactttttaaatgatatgaggtagagctgggacatctgccctttcctgtggacttaaaaaaccaaaacaaaactatgattggcatcttccaaaagtgatttgaaaaacatgatgttgcccctctaacaaagcattgataaggttaagaatttggtttacattgtgtctatgtatctgggaatcatctctgggaggtcaagatgtactgttctacccgttttacagatgacatggagggattcaagggagagtggctgcaaagtcacgtagagcgtcagtgtaaagctgggaatcaatttgtggttcaagcttgtgacccaaactcctccctatgtttcctcattttggataaattagccagtttccaagaaagaggccctgagctgaagggtgagcgttggtcccagtgaagggtgagaccccttcactgcctcttctgcagcccttttcctcctcaagtctctgggagccctctggggttatcactgacggatccattaagttccttcatattcaattatacctggcctttttagagacatttaatttaaagtggagataacactctcaaacaaagttaaaatcctattgggctaagaggagctgtttgagtgatgaagaggaagagagctattcagcaccccagcagatcacattacgtagtgactgtgggctcttccccctgaggcctgcccacttggtaaccaatgaagtgctgtctctgatcttgtcactccctggcccaaaaaccttgaatgtccacacactactacagattcaataactaactttcaaggtgctcagcaatatggcgtctgcctgctttcctggagacagcacattttcttactctggccttggtaagtgactttcaaaggttttatcaaatagcccttatggatctcattttgttccttccctcatatcccttctccttcccatctgtcattatcatatttattcctgatgcctatctgcagtgccagctccctttctgggccttttttgacttgcaggtaagcccttgactatgctctacttttcgtcttacttcctcccccaccacacgcgtgatttaaattttttcaggacagaggttcattcttataaccttcacagcttttgtcaagatgtcgtgtatgaacaaggcattcaatacacatttgttggttgactgggatggacctccccctggagctgtagatcctccagcctaatggaaggccatttagaatcacacttgcactgtgagtggacactgccattgggaaaaatagccttctctttggggacccagagggtaacctgctcttgcttaggtacaattacggccctgtgaatggaattgggtcatagtgatgaaatctccaaattggatgaaactactctatcaaagtagttttcttttgcctcattcaggggcttgagccctactagcccaatgaaaatcgggttttgctaagtagactttgcctgtcaattggcagcaaattcacctggggcacttggcacctcctcctgttcagggactggcctggcagggcctctccctgttcgcatctagtgtctgggctatttgaagccctctctgtgccaaatcctcaaactcctgcttccgttcgattcagcccatcttctcttctttttaaaaactgatgaatgtctttaattggatcatggtcacccataggaggtcaggaactgtgctctcactggaaagatggaaacaccaaaaccgttaaagaacaagattctccctgatgttagccagctttcattcatgtcttgactgtgttatgaaaagggaggttacctatagaaaataaataaaagaatgagattcattttcccagcaatctgaaagtttctgcgctataaagcacttgattttttggtgggggggatcttaactgaaagcatgtctgaaaataaggatgttcatgatgacaggctggctggatttacatttgaaggttgttgaaaatagctattcctcataatctgggtatagagttgccagatttagcaaacaaacaaacagacaaacaaaataaaacaaaaccaatcccctccccacagaaacccaaactgaaataaaaccagaaaaccaggaagcccaggtaaatttgaatttaagataaataataaataaatttttagcataagtctgtctgtctcatacagtatttgggatgacttatactaaaaaattatgtatctgaaaatgaaattttatggggcgtttggtctgcctaggttcccagagtactaatggtaagaggacttaaagcaaatacgggaaggtaggagaaaacagttgaggacaaattcagctcttctggtctttgtcaaaggcaaggctggccgggcgtggtggctaacacctgtaatctcagcactttgggaggctgtggtgggtggataatgaggtcaggagttcgagaccagcctggccagtttttagtaaagaggtgagtaaaaccctgtctctactaaaaatacaaaaattagccgggcatggtggtatgcacctgtagtcccagctacttgggaggctgaggcagaagacttgcttgaacccaggaggtggaggttacagtgagccaagatcatgccactatactccagcctggcgacagagtgagactccatctcaaaaaaaaaaaaaaaaagaaaaaagaaaaaaaaaaggtaaggctgctattttcatgacattcatgcaagaacatcttgagttacatatgtatatatattcttttttgcctagaacaaagaagaaccaaaaagcaaaggtactgtcatttgaaagcttgttattatttacattactttcttataataattgcactaataagaacaatggattggctgggcgtggtggctcacgcctgtaatcccagcactttgggaggccgaggcaggcagatcacgaggtcaggaaatcgagaccatcctggctaacatggtgaaaccctgtctctactaaaaatacaaaaaatgagccaggcgtggtggtgggtgcctgtagtcccgggaggctgaggcaggagaatggcgtgaacccgggaggcggagattgcaatgagctgagattgcgccactgaactccagcctgggagacagcaagactccgtctcaaaaaaaaaaaaaaatggattgcattttttgaacatttactttgttctagacattgtgcattgcgtatatcatcttaccttatctctcaaacaatggtgggaggtagctattttgttttacagaggaggaaacttgagtcttcaggaagttaagtggattttccaaggtctccagcaagtggcagaacagggactcaagctccttagttctgactgcagggctcgagattttaactccagctaggtgctgatattttttctgatctgtgtgttctgtttatcaaaattgtctttgaacttaagatttataaaaggtgaaggaaggaaatgaatctttttgatgatcagaacagtgcacagagtattcgggaacctgtcttgtaatgttttctttcattgattcaatgacaaatagttattgaaactctcccagggtctgttttgggtacttgaggcacagtgggcaaaaatctctgtcctaaaagagcttactttctagagtgggaggaatatcacacgaatgaaaggtagactacgtcgtgtggtattgatcagtgctgtggtggaaaataaagcaagatgggggatgggaagtttctgggcatggagatggaatgttgcaattttaaataggatggtcaggaaatgcttccctgagagggtgacattctaacaaaaacccaaggttggtgaaagagtgaatcatacgggagaagaatgttccaggcagaaggaacagtaagtgcaaaggccctgagctggggctgttcctggtgggtcagaggagcaataaggagaccgccgtgagcctagtgaggaagtcagtgaggtgggaatggttgcaggcatttcagaaggtagagttgcagagaaggtgatgtaggtcttgaaggtgatcataaggtctttgatgtttgttctgagtgagatgggaaatcactggggctttgggcagaggagtaacatgatctgacttaggtttaaacaggatcactcagggccgctgtgttgcaaatagattgtagggagtaaaaatggaagaggggagaccagttagaaggtatttgcaatgactaagatgattcatttgctgactatgcatggagcacttgctgtgtgctatggtctctcctgggagcttagaatatggtcttgagtgaaatcagcttcttgctttcaggagtttgttttctactgggagacgacagagcaacaagtaaatcaacgaataacaagttaatttctgatagtgataaatgatactaaaaaactgaaacaagatcatatgttctaatgaattctctgtttctatctatggggacagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccacctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaacaaaacccatgtactagtcttccttaccccagaggaattccagctgcaagcttctttagggaaaatgctcccttccccttttaactgagcaattatctacacaagaaataagactgctcagatatacaaagagagtagcttcaatgaaaagatgtttggatttggataattcttttccctagcaaaattcgctagctcccttaagagtcttaataaagaggctacgttgggattaaaagaaaaaaaaacagaaataaaatat

SEQ ID NO: 8 - DNA, 22 bp, Homo sapiens
tcagaagaaaatcagaggaatc

SEQ ID NO: 9 - DNA, 23 bp, Homo sapiens
gggacgttcagtagcggttgtag

SEQ ID NO: 10 - DNA, 20 bp, Homo sapiens
ctgcccatggacaccgagac

SEQ ID NO: 11 - DNA, 23 bp, Homo sapiens
ccgtttgcatatactcttctgag

(19) United States
(12) Patent Application Publication
(10) Pub. No.: US 2007/0014736 A1
(43) Pub. Date: Jan. 18, 2007

(54) Methods of delivering corticotroph-derived glycoprotein hormone

(76) Inventors: Shannon L. Okada, Seattle, WA (US); Katherine E. Lewis, Lake Forest Park, WA (US); Jeff L. Ellsworth, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 11/472,878

(22) Filed: Jun. 21, 2006

Related U.S. Application Data
(60) Provisional application No. 60/695,147, filed on Jun. 29, 2005.

(51) Int. Cl. A61L 9/04; A61K 38/17

(52) U.S. Cl. 424/45; 514/12

(57) ABSTRACT

Methods for treating diseases comprising delivering CGH by aerosol delivery and comprising administering a lesser amount of CGH than TSH are described.

### CROSS REFERENCE TO RELATED APPLICATIONS

This application claims the benefit of U.S. Provisional Application Ser. No. 60/695,147, filed Jun. 29, 2005, which is herein incorporated by reference.

### BACKGROUND OF THE INVENTION

Aerosol delivery offers an alternative route of many drugs, and can be especially useful for therapeutic proteins. Many therapeutic proteins are delivered by injection, which encounters problems such as patient compliance and overall safety concerns regarding inadvertent needle sticks. Other methods of delivery, such as nasal or pulmonary administration of the therapeutic protein, offer a convenient alternative to injections.

There are limitations, however, on the sorts of proteins that lend themselves to aerosol delivery. For example, high molecular weight proteins may not pass the mucosal barrier and may not provide for systemic delivery. Thus, some proteins that are difficult to deliver require other molecules to aid in absorption or delivery of the drug. See, in general, Arora, P., et al, Drug Discovery Today 7:967-975, 2002.

In addition, many large therapeutic proteins require large amounts of protein to be delivered to be efficacious. Such large amounts of protein can be difficult and costly to manufacture. The present invention provides methods of delivering Corticotroph-derived Glycoprotein Hormone (CGH) by aerosol delivery and methods for treating diseases by delivering lesser amounts of CGH.

### DETAILED DESCRIPTION OF THE INVENTION

The present invention provides novel methods for delivering a previously described protein, Corticotroph-derived Glycoprotein Hormone (CGH, also called Thyrostimulin). Corticotroph-derived glycoprotein hormone (CGH) is a heterodimeric protein hormone released from corticotroph cells in the anterior pituitary. CGH is disclosed in International Patent Application No. PCT/US01/09999, publication no. WO 01/73034, herein incorporated by reference. It is comprised of an alpha subunit, glycoprotein hormone alpha2 (GPHA2), and a beta subunit, glycoprotein hormone beta 5 (GPHB5). GPHA2 was previously called Zsig51 (See International Patent Application No. PCT/US99/03104, publication no. WO 99/41377 published Aug. 19, 1999; U.S. Pat. No. 6,573,363, herein incorporated by reference). SEQ ID NO: 1 is the human cDNA sequence that encodes the full-length polypeptide GPHA2, and SEQ ID NO:2 is the full-length polypeptide sequence of human GPHA2. SEQ ID NO:3 is the mature GPHA2 polypeptide sequence without the signal sequence. Thus, the mature GPHA2 polypeptide comprises or consists of the amino acid sequence of residues 24 to 129 of SEQ ID NO: 2. GPHB5 was previously called Zlut1 (See International Patent Application No. PCT/ US02/22747, publication no. WO 03/006051 published Jan. 23, 2003). SEQ ID NO: 4 is the human cDNA sequence that encodes the full-length GPHB5 polypeptide. SEQ ID NO: 5 is the full-length GPHB5 polypeptide. SEQ ID NO: 6 is the mature GPHB5 polypeptide without the signal sequence. Thus, the mature GPHB5 polypeptide comprises or consists of the amino acid sequence of residues 25 to 130 of SEQ ID NO: 5. SEQ ID NO: 7 is the human genomic DNA sequence that encodes the full-length GPHB5 polypeptide. The present invention also includes CGH polypeptides, and polynucleotides, that are substantially homologous to those of the SEQ ID NOs: 1, 2, 3, 4, 5, 6, and 7.

CGH and methods of using it to treat diseases have been previously described in published patent applications for indications including treating metabolic disorders, including diabetes and lypolysis (See International Patent Application No. 20003006051, published Jan. 23, 2003); treating inflammation (See International Patent Application No. 03/104807, published Dec. 18, 2003); and treating liver steatosis (See International Patent Application Publication No. 2005/087256, filed Mar. 4, 2005), all of which are herein incorporated by reference.

The present invention shows that recombinantly produced CGH protein binds and activates the thyroid-stimulating hormone receptor (TSHR) and is a more potent competitor for the TSHR than TSH. Analysis of rhCGH binding to the TSHR, moreover, indicates that CGH binds a separate binding domain on the TSHR than TSH. Thus, one site is recognized by and binds CGH but not TSH, and another site is recognized by and binds both hormones. The thyroid-stimulating properties of CGH were confirmed by the elevations of thyroxine (T4) observed following a single intraperitoneal dose of rhCGH in mice. See also Okada, S. L., et al., Mol Endocrinol. 20(2):414-25, 2006.

The present invention shows that the administration of the CGH heterodimer intranasally results in systemic administration of the CGH heterodimer. Thus, even though the CGH heterodimer is a large protein having a moleucular weight of about 34 kD, systemic administration of the protein can be achieved by aerosol administration, including, but not limited to administration by intranasal and/or pulmonary inhalation.

The teachings of all of the references cited herein are incorporated in their entirety herein by reference.

CGH exerts its effects through interaction with the thyroid-stimulating hormone (TSH), or thyrotropin, receptor. The TSH receptor (TSHR) is a member of the G-protein coupled, seven-transmembrane receptor superfamily. Activation of the TSH receptor leads to coupling with heterotrimeric G proteins, which evoke downstream cellular effects. The TSH receptor has been shown to interact with G proteins of subtypes Gs, Gq, G12, and Gi. In particular, interaction with Gs leads to activation of adenyl cyclase and increased levels of cAMP. See Laugwitz et al., Proc Natl Acad Sci USA 93: 116-20 (1996). Elevation of cAMP levels leads to activation of protein kinase A, a multi-potent protein kinase and transcription factor eliciting diverse cellular effects. See Bourne et al., Nature 349: 117-27 (1991).

The TSHR was originally identified in the thyroid as the principal activator of the thyroid gland, following exposure to the glycoprotein hormone, TSH. TSH release from the anterior pituitary stimulates the TSHR, resulting in secretion of thyroid hormone, stimulation of thyroid hormone synthesis, and cellular growth. TSH release is regulated by thyroid hormone levels, and is potently inhibited by elevated glucocorticoid levels. See, Utiger, in Endocrinology and Metabolism (Felig and Frohman, eds), pp. 261-347, McGraw-Hill, (2001).

Recently, the TSHR has been identified in many cell types not previously recognized, including cells of the immune system, brain, adipose, and reproductive organs. See, Example 3. These tissues are also targets of glucocorticoid action, suggesting a coordinate role for CGH and GC's as effectors of adrenal functions.

Methods of expressing the recombinant CGH molecules of the present invention are well-known and include expression vectors comprising such nucleic acid molecules, recombinant host cells comprising such vectors and expression vectors, and recombinant viruses comprising such expression vectors. These expression vectors and recombinant host cells can be used to prepare CGH polypeptides. In addition, the present invention provides pharmaceutical compositions, comprising a pharmaceutically acceptable carrier and at least one of such an expression vector or recombinant virus. Preferably, such pharmaceutical compositions comprise a human CGH gene, or a variant thereof.

The present invention further contemplates antibodies and antibody fragments that specifically bind with CGH polypeptides. Such antibodies include polyclonal antibodies, murine monoclonal antibodies, humanized antibodies derived from murine monoclonal antibodies, and human monoclonal antibodies. Examples of antibody fragments include F(ab′)2, F(ab)2, Fab′, Fab, Fv, scFv, and minimal recognition units.

CGH can be administered in conjunction with or in place of glucocorticoid treatment. This means that CGH is administered before, during or after administration of the steroid, as well as a stand-alone therapy. Treatment dosages should be titrated to optimize safety and efficacy. Methods for administration include intravenous, peritoneal, intramuscular, and topical. Pharmaceutically acceptable carriers include but are not limited to, water, saline, and buffers. Dosage ranges would ordinarily be expected from 0.1 μg to 0.1 mg per kilogram of body weight per day, with the exact dose determined by the clinician according to accepted standards, taking into account the nature and severity of the condition to be treated, patient traits, etc. Within this dosage range, a dose of 5 μg/kg/day can be used. Also within this range, a range from 5 μg/kg/day to 100 μg/kg/day can also be used. A useful dose to try initially would be 40 to 50 μg/kg per day. However, the doses may be higher or lower as can be determined by a medical doctor with ordinary skill in the art. For a complete discussion of drug formulations and dosage ranges see Remington's Pharmaceutical Sciences, 17th Ed., (Mack Publishing Co., Easton, Pa., 1990), and Goodman and Gilman's: The Pharmacological Bases of Therapeutics, 9th Ed. (Pergamon Press 1996).

For pharmaceutical use, the proteins of the present invention can be administered as a pulmonary or nasal inhalant. In general, pharmaceutical formulations will include a CGH protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Doses of CGH polypeptide will generally be administered on a daily to weekly schedule. Determination of dose is within the level of ordinary skill in the art. The proteins may be administered for acute or chronic treatment, over several days to several months or years. In general, a therapeutically effective amount of CGH is an amount sufficient to produce a clinically significant change in an inflammatory condition, a metabolic disorder, or liver steatosis.

The invention provides a method of delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery. The delivery can be intranasal inhalation or pulmonary inhalation. The first protein can consist of the amino acid sequence from residue 24 to residue 129 of SEQ ID NO: 2. The second protein can consist of the amino acid sequence from residue 25 to 130 of SEQ ID NO: 5. Further, the heterodimeric protein can comprise or consist of a first protein consisting of the amino acid sequence from residue 24 to residue 129 of SEQ ID NO: 2, and the second protein can consist of the amino acid sequence from residue 25 to 130 of SEQ ID NO: 5. This protein can be delivered by intranasal inhalation or pulmonary inhalation.

The invention also provides a method of reducing the amount of a heterodimeric protein delivered, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, wherein the heterodimeric protein is delivered by aerosol inhalation, and wherein a lesser amount of protein is necessary than by oral, intraperitoneal, intramuscular, or subcutaneous delivery. The first protein can consist of the amino acid sequence from residue 24 to residue 129 of SEQ ID NO: 2. The second protein can consist of the amino acid sequence from residue 25 to 130 of SEQ ID NO: 5. Further, the heterodimeric protein can comprise or consist of a first protein consisting of the amino acid sequence from residue 24 to residue 129 of SEQ ID NO: 2, and the second protein can consist of the amino acid sequence from residue 25 to 130 of SEQ ID NO: 5. This method of reducing the amount of heterodimeric protein delivered can be delivered by intranasal inhalation or pulmonary inhalation.

The invention provides a method of reducing inflammation in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein inflammation is reduced. The delivery can be by intranasal inhalation or pulmonary inhalation. The first protein can consist of the amino acid sequence from residue 24 to residue 129 of SEQ ID NO: 2. The second protein can consist of the amino acid sequence from residue 25 to 130 of SEQ ID NO: 5. Further, the heterodimeric protein can comprise or consist of a first protein consisting of the amino acid sequence from residue 24 to residue 129 of SEQ ID NO: 2, and the second protein can consist of the amino acid sequence from residue 25 to 130 of SEQ ID NO: 5. This protein can be delivered by intranasal inhalation or pulmonary inhalation.

The invention provides a method of inducing lypolysis in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein lypolysis is induced. The delivery can be by intranasal inhalation or pulmonary inhalation. The first protein can consist of the amino acid sequence from residue 24 to residue 129 of SEQ ID NO: 2. The second protein can consist of the amino acid sequence from residue 25 to 130 of SEQ ID NO: 5. Further, the heterodimeric protein can comprise or consist of a first protein consisting of the amino acid sequence from residue 24 to residue 129 of SEQ ID NO: 2, and the second protein can consist of the amino acid sequence from residue 25 to 130 of SEQ ID NO: 5. This protein can be delivered by intranasal inhalation or pulmonary inhalation.

The invention provides a method of reducing liver steatosis in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein liver steatosis is reduced. The delivery can be by intranasal inhalation or pulmonary inhalation. The first protein can consist of the amino acid sequence from residue 24 to residue 129 of SEQ ID NO: 2. The second protein can consist of the amino acid sequence from residue 25 to 130 of SEQ ID NO: 5. Further, the heterodimeric protein can comprise or consist of a first protein consisting of the amino acid sequence from residue 24 to residue 129 of SEQ ID NO: 2, and the second protein can consist of the amino acid sequence from residue 25 to 130 of SEQ ID NO: 5. This protein can be delivered by intranasal inhalation or pulmonary inhalation.

The invention is further illustrated by the following non-limiting examples.

### EXAMPLE 1

CGH is Expressed in Corticotrophs

Summary: The cell-specific localization of CGH expression in the anterior pituitary was evaluated in two stages. First, double in situ hybridization was used to demonstrate the co-expression of GPHA2 and GPHB5 mRNAs in the same subset of cells in the anterior pituitary. Next, the identity of these cells was evaluated using double immunohistochemical methods to stain for the localization of GPHB5 protein relative to protein markers for different cell populations in the anterior pituitary. These markers included growth hormone (a marker for somatotrophs), follicle-stimulating hormone (gonadotrophs), luteinizing hormone (gonadotrophs), thyroid-stimulating hormone (thyrotrophs), adrenocorticotrophic hormone (corticotrophs), prolactin (mammotrophs) and S-100 protein (follicular stellate and dendritic cells). GPHB5 protein was found to only co-localize with adrenocorticotrophic hormone, showing that it was produced by corticotrophs. GPHB5 protein was not found to co-localize with any of the other markers. Taken together, the immunohistochemical data and the in situ data described above show that the heterodimeric glycoprotein hormone CGH is produced by corticotrophs.

A. Identification of Cells Expressing GPHA2 and GPHB5 Using in situ Hybridization.

Human pituitaries were screened for GPHA2 and GPHB5 expression by in situ hybridization. The tissues were fixed in 10% buffered formalin and embedded in paraffin blocks using standard techniques. Tissues were sectioned at 4 to 8 microns, and the sections were prepared using a standard protocol. Briefly, tissue sections were deparaffinized with HistoClear (National Diagnostics, Atlanta, Ga.) and then dehydrated with ethanol. Next they were digested with Proteinase K (50 μg/ml) (Boehringer Diagnostics, Indianapolis, Ind.) at 37° C. for 3 to 10 minutes. This step was followed by acetylation and re-hydration of the tissues.

Using oligonucleotides specific for GPHB5 sequences, a polymerase-chain-reaction-based in situ method was used to visualize GPHB5 mRNA with a FITC detection system, which gives a green signal. Following this reaction, the same slide was subjected to a standard in situ hybridization protocol using a probe designed against the human GPHA2 sequence. T7 RNA polymerase was used with a linearized plasmid template containing the entire coding domain and the 3′UTR of GPHA2 to generate an antisense probe. The probe was labeled with digoxigenin (Boehringer, Ingelheim, Germany) using an In Vitro Transcription System kit (Promega, Madison, Wis.) following the manufacturer's instructions. The digoxigenin-labeled GPHA2 probe was added to the slides at a concentration of 1 to 5 pmol/ml for 12 to 16 hours at 60° C. Slides were subsequently washed in 2×SSC and 0.1×SSC at 55° C. The signals were amplified using tyramide signal amplification (TSA, in situ indirect kit; NEN, PerkinElmer Life Sciences, Boston, Mass.) and visualized with Texas Red following the manufacturer's instructions. The slides were then counter-stained with hematoxylin (Vector Laboratories, Burlingame, Calif.) and evaluated microscopically.

Results: A subset of scattered cells in the anterior pituitary show both green and red signal, indicating that they were positive for both GPHB5 and GPHA2 mRNA expression. There are few or no cells that express only one of the two messages.

B. Immunohistochemical Double Staining of GPHB5 vs. Markers for Anterior Pituitary Cell Types

Human anterior pituitaries were screened using antibodies against GPHB5 and a variety of cell-type-specific markers to determine which cell types express GPHB5 protein. Double immunostains were performed for GPHB5 vs. growth hormone (GH; a marker for somatotrophs), follicle-stimulating hormone (FSH; gonadotrophs), luteinizing hormone (LH; gonadotrophs), thyroid-stimulating hormone (TSH; thyrotrophs), adrenocorticotrophic hormone (ACTH; corticotrophs), prolactin (PRL; mammotrophs) and S-100 protein (follicular stellate and dendritic cells).

Sandwich technique immunohistochemistry was applied in this study, using two primary antibodies (anti-GPHB5 and antibodies against one of the marker proteins) and two detection systems: immunoperoxidase (IP) with Diaminobenzidine (DAB) (Ventana Bio Tek, Tucson, Ariz.), leading to a brown signal indicating the presence of GPHB5, and alkaline phosphatase (AP) with BioTek Red, (Ventana Bio Tek) leading to a red signal indicating the presence of the marker protein in question.

The experiments were performed on sections of a human pituitary gland taken from a 24-year-old male who died of a gunshot wound (tissue block internal reference number H01.2075). The tissue was fixed in 10% buffered formalin and embedded in paraffin blocks using standard techniques. The tissue was sectioned at 4 to 8 microns, and the sections were prepared using a standard protocol.

Reagents and Protocol:

Normal goat blocking serum (ChemMate, CMS/Fisher: Cat #: 028-337).

Primary antibodies: a) Rabbit anti-human GPHB5 protein (produced in-house, internal reference number E3039), working dilution: 1:3200. b) Rabbit anti-human GH (Zymed Laboratories, South San Francisco, Calif.) Cat. No. 18-0090), working dilution: 1:25. c) Mouse anti-human FSH (Zymed, Cat. No. 18-0020), working dilution: 1:50. d) Mouse anti-human LH (Zymed, Cat. No. 18-0037), working dilution: 1:50. e) Mouse ant-human TSH (Zymed, Cat. No. 18-0051), working dilution: 1:50. f) Rabbit anti-human ACTH (Zymed, Cat. No. 18-0087), working dilution: 1:50. g) Rabbit anti-PRL (Zymed, Cat. No. 18-0086), working dilution: 1:50. h) Rabbit anti-S-100 protein (Zymed, Cat. No. 18-0046), working dilution: 1:1000 and 1:2000.

Secondary antibodies: a) Biotinylated goat anti rabbit IgG (Vector, Cat. No: BA-1000), working solution: 7.5 μg/l, diluted in PBS with 2% normal goat serum. b) Biotinylated goat anti mouse IgG (Vector, Cat. No: BA-9200), working solution: 7.5 μg/l, diluted in PBS with 2% normal goat serum and 2% non-fat dried milk.

Detection reagents: a) DAB Detection Kit (Ventana Bio Tek Systems, Tucson, Ariz. Catalog No: SDK2502). b) AP Detection Kit (Ventana Catalog No: SDK306).

Method: TechMate 500 autoimmunstainer (Biotech/Ventana), IP-AP protocol with modifications. Avidin/Biotin block following heat-induced epitope retrieval.

Summary of Results:

Positive staining was seen for GPHB5 and all other primary antibodies. GPHB5 was found to co-localize only with ACTH, and not with FSH, GH, LH, PL and TSH. GPHB5/S-100 staining was less than optimal, but GPHB5 and S-100 co-localization was not indicated. GPHB5 staining was seen in the majority of ACTH-producing pituicytes. There are few if any cells producing GPHB5 that do not also express ACTH.

### EXAMPLE 2

CGH Activation of Adrenal Cortex Cells Results in cAMP Production

Summary: A human adrenal cortex cell line, NCI-H295R, was used to study signal transduction of CGH. NCI-H295R was transduced with recombinant adenovirus containing a reporter construct, a firefly luciferase gene under the control of cAMP response element (CRE) enhancer sequences. This assay system detects cAMP-mediated gene induction downstream of activation of Gₛ-coupled GPCR's (G-protein coupled receptors). Treatment of NCI-H295 with purified CGH heterodimer protein produced a dose-dependent induction of luciferase activity equal to or higher than that induced by 10 μM forskolin, a constitutive inducer of adenyl cyclase. Typically, CGH elicited a maximal response of 15-40-fold luciferase induction above control media. These results demonstrate CGH signaling through a GPCR in adrenal cortical cells and the production of cAMP.

Experimental Procedure.

NCI-H295R cells were obtained from the ATCC (CRL-2128, Manassas, Va.) and cultured in growth medium as follows: 1:1 mixture of Dulbecco's modified Eagle's medium and Ham's F12 medium with L-glutamine (D-MEM/F-12; GIBCO, cat.#11320-033) containing 25 mM HEPES buffer (GIBCO, Invitrogen, Carlsbad, Calif., cat.#15630-080), 1 mM sodium pyruvate (GIBCO, cat.#11360-070), 1% ITS+1 media supplement (Sigma St. Louis, Mo. cat#I2521) and 2.5% Nu-Se I (BD Biosciences, Lexington, Ky. cat.#355100). Cells were cultured at 37° C. in a 5% CO₂ humidified incubator. One or two days before assaying, cells were seeded at 20,000 cells per well in a 96-well white opaque/clear bottom plate (BD Biosciences, cat.#356650). One day before assay, cells were transduced with AV KZ55, an adenovirus vector containing KZ55, a CRE-driven luciferase reporter cassette, at 5000 particles per cell. Following overnight incubation, the cells were rinsed once with assay medium (D-MEM/F-12 supplemented with 0.1% bovine serum albumin, ICN Biomedicals, Inc., Aurora, Ohio, cat.#103700), followed by incubation for four hours at 37° C. in assay medium to which test protein had been added. The plate was then washed with phosphate buffered saline (GIBCO, cat. #20012-027).

Promega's Luciferase Assay System (Promega, Madison, Wis., cat. #E1500) was used to process the treated cells. Cell lysis buffer, 25 μl/well, was added to each well and incubated at room temperature for 15 minutes. Luciferase activity was measured on a microplate luminometer (PerkinElmer Life Sciences, Inc., model LB 96V2R) following automated injection of luciferase assay substrate.

### EXAMPLE 3

Distribution of TSH Receptor Gene Expression.

We surveyed RNA samples for TSHR transcript using reverse transcriptase polymerase chain reaction (RT-PCR) amplification. Using standard procedures, RNA samples were isolated from tissues and cell lines, and RT-PCR was run with two separate pairs of primers. The first primer pair includes the forward primer (5′TCAGAAGAAAATCAGAGGAATC) (SEQ ID NO:8) and the reverse primer (5′GGGACGTTCAGTAGCGGTTGTAG) (SEQ ID NO:9), which amplify a 487 bp product. The amplified product spans an intron to control for signal arising from genomic DNA contamination. The second primer pair includes the forward primer (5′CTGCCCATGGACACCGAGAC) (SEQ ID NO:10) and the reverse primer (5′CCGTTTGCATATACTCTTCTGAG) (SEQ ID NO:11) and amplifies a 576 bp product. Additionally, TSHR expression was assessed from data in the published literature. Results are described below.

A. TSH Receptor in Immune Related Cells.

TSH-R is expressed in human CD14+ monocytes (decreasing expression after activation), in the human monocytic cell lines THP-1 and PMA-activated HL60 (but not in U937), in resting (but not activated) human NK cells, in human “resting” CD3+ (primarily CD4+) T cells, and in human B cells and B cell lines. Among mouse immune cell subsets, we have found that mTSH-R is expressed in CD4+ but not CD8+ T cells (decreasing with activation), in B cells (decreasing slightly with activation), and in an IFN□-activated mouse macrophage line, J774.

Additionally, TSHR transcript has also been shown to be present in lymphocytes (Szkudlinski M. W., Fremont V., Ronin C., Weintraub B. D., (2002) Physiol Rev 82: 473-502) and other immune related cell types (Bagriacik E U, and Klein J R, (2000) J Immunol 164: 6158-65).

B. TSH Receptor in Adrenal Gland.

RNA from the adrenal cortex carcinoma cell line H295R along with RNA isolated from several adult human normal adrenal glands were found positive for TSHR. Published literature also documents TSHR transcript in the adrenal gland (Dutton C. M., Joba W., Spitzweg C., Heufelder A. E., Bahn R. S., (1997) Thyroid 6: 879-84).

C. TSH Receptor in a Wide Variety of Cells and Tissue Types.

Extensive panels of RNAs were screened for TSHR and positive expression was found in thyroid, adrenal gland, kidney, brain, skeletal muscle, testis, liver, osteoblast, aortic smooth muscle, ovary, adipocytes, retina, salivary gland, and digestive tract. Similarly, the published literature documents TSHR expression in thyroid, kidney, thymus, adrenal gland, brain, retroocular fibroblasts, neuronal cells and astrocytes (Szkudlinski M. W., Fremont V., Ronin C., Weintraub B. D.,(2002) Physiol Rev 82: 473-502 and Dutton C. M., Joba W., Spitzweg C., Heufelder A. E., Bahn R. S., (1997) Thyroid 6: 879-84).

### EXAMPLE 4

In vivo Stimulation of Adrenal Cortex by CGH.

Summary: Mice were exposed to CGH through infection with adenovirus particles expressing GPHA2 and GPHB5, leading to overexpression and secretion of CGH from the liver of these animals. Profound adrenal hypertrophy and vacuolization were observed in mice sacrificed three weeks after adenoviral infection. The hypertrophy was apparent in the inner cortical layers, the zona fasciculata and the zona reticularis. Similarly, mice were exposed to CGH through intraperitoneal injection of recombinant CGH protein alone, recombinant CGH protein along with the glucocorticoid Dexamethasone (Dex), Dex alone, or PBS alone daily for two weeks. Significant gain in adrenal weight was demonstrated in female mice after chronic treatment with CGH or CGH along with Dex.

A. Generation of GPHB5 and GPHA2 Expressing Recombinant Adenovirus.

The protein coding regions of GPHA2 and GPHB5 were amplified by PCR using primers that added FseI and AscI restriction sites at the 5′ and 3′ termini respectively. PCR primers were used with the templates containing the full-length GPHA2 and GPHB5 cDNAs in standard PCR reactions. The PCR reaction products were loaded onto a 1.2% (low melt) SeaPlaque GTG (FMC, Rockland, Me.) gel in TAE buffer. The products were excised from the gel and purified using the QIAquick®PCR Purification Kit gel cleanup kit as per kit instructions (Qiagen, Valencia, Calif.). The PCR products were then digested with FseI-AscI, phenol/chloroform extracted, EtOH precipitated, and rehydrated in 20 uL TE (Tris/EDTA pH 8). The products were then ligated into the FseI-AscI sites of the vector pMT 12-8 and transformed into DH10B cells by electroporation. Clones containing the appropriate inserts were identified by plasmid DNA miniprep followed by digestion with FseI-AscI, and the constructions verified by DNA sequencing. DNA was prepared using a commercially available kit (Qiagen, Inc.) The GPHA2 and GPHB5 cDNAs were released from the pMT12-8 vector using FseI and AscI enzymes. The cDNAs were isolated on a 1.2% low melt gel, the gel slices melted at 70° C., extracted twice with an equal volume of Tris-buffered phenol, and EtOH precipitated. The DNAs were resuspended in 10 uL of water.

The GPHA2 and the GPHB5 recombinant adenoviruses were prepared using different vectors. The GPHA2 cDNA was ligated into pACCMV shuttle vector (Microbix Biosystems, Inc. Ontario, Canada) in which the polylinker had been modified to include FseI and AscI sites and transformed into E. coli host cells (Electromax DH10B™ cells; obtained from Life Technologies, Inc., Gaithersburg, Md.) by electroporation. Clones containing GPHA2 inserts were identified by plasmid DNA miniprep followed by digestion with FseI and AscI. A large-scale preparation of DNA was made for transfection. The GPHA2-containing shuttle vectors were co-transfected with E1-deleted, adenovirus vector pJM17 (Microbix Biosystems, Inc.) into 293A cells (Quantum Biotechnologies, Inc. Montreal, QC. Canada) that express the adenovirus E1 gene. The DNA was diluted up to a total volume of 50 ul with sterile HBS (150 mM NaCl, 20 mM HEPES). In a separate tube, 20 uL DOTAP (Boehringer-Ingelheim, 1 mg/ml) was diluted to a total volume of 100 ul with HBS. The DNA was added to the DOTAP, mixed gently by pipeting up and down, and left at room temperature for 15 minutes. The media was removed from the 293A cells and washed with 5 ml serum-free MEMalpha containing 1 mM sodium pyruvate, 0.1 mM MEM non-essential amino acids and 25 mM HEPES buffer (all from Life Technologies, Inc.). 5 ml of serum-free MEM was added to the 293A cells and held at 37° C. The DNA/lipid mixture was added drop-wise to the T25 flask of 293A cells, mixed gently, and incubated at 37° C. for 4 hours. After 4 hours the media containing the DNA/lipid mixture was aspirated off and replaced with 5 ml complete MEM containing 5% fetal bovine serum.

The 293A cells were maintained for 24 weeks before recombination of the endogenous viral sequences and the transfected viral vector resulted in the production of infectious viral particles. Within 5 days of recombination, propagation of infectious virus produced lysis of the culture monolayer. The medium containing the viral lysate was collected and any remaining intact cells were lysed by repeated freeze/thaw cycles and the cell debris was pelleted by centrifugation.

The viral lysate was then plaque-purified according to the method of Becker et al., Meth. Cell Biol. 43:161-189, 1994. Briefly, serial dilutions were prepared in DMEM containing 10% fetal bovine serum and 100 U/ml penicillin/streptomycin, added to monolayers of 293 cells, and incubated at 37° C. for one hour. A melted 1.3% agarose/water solution was mixed with 2× DMEM (containing 4% FBS, 200 U/ml penicillin/streptomycin, 0.5 ug/ml fungizone and 30 mg/ml phenol red), and 6 ml of the mixture was added to the virus-infected 293 cells. Plaques were visible within 7-10 days. Single plaques were isolated, and the presence of the GPHA2 insert was verified by PCR. One plaque that had the expected size PCR product was used to do a primary amplification.

The GPHB5 adenoviral construction was produced in a second vector system, pAdTrack CMV (He, T-C. et al., PNAS 95:2509-2514, 1998). This vector contains the Green Fluorescent Protein (GFP) marker gene, and was first modified by replacing the promoter and polyadenylation sequences of the GFP gene with SV40 and human growth hormone sequences, respectively. In addition, the native polylinker was replaced with FseI, EcoRV, and AscI sites. This modified form of pAdTrack CMV was named pZyTrack. Ligation was performed using the Fast-Link® DNA ligation and screening kit (Epicentre Technologies, Madison, Wis.). Clones containing GPHB5 were identified by digestion of mini prep DNA with FseI-AscI. In order to linearize the plasmid, approximately 5 μg of the pZyTrack GPHB5 plasmid was digested with PmeI. Approximately 1 ug of the linearized plasmid was cotransformed with 200 ng of supercoiled pAdEasy (He et al., supra.) into BJ5183 cells. The co-transformation was done using a Bio-Rad Gene Pulser at 2.5 kV, 200 ohms and 25 mFa. The entire co-transformation was plated on 4 LB plates containing 25 ug/ml kanamycin. The smallest colonies were picked and expanded in LB/kanamycin and recombinant adenovirus DNA identified by standard DNA miniprep procedures. Digestion of the recombinant adenovirus DNA with FseI-AscI confirmed the presence of GPHB5. The recombinant adenovirus miniprep DNA was transformed into DH10B competent cells and DNA prepared using a Qiagen maxi prep kit as per kit instructions.

Approximately 5 ug of recombinant adenoviral DNA was digested with PacI enzyme (New England Biolabs, Beverly, Mass.) for 3 hours at 37° C. in a reaction volume of 100 uL containing 20-30 U of PacI. The digested DNA was extracted twice with an equal volume of phenol/chloroform and precipitated with ethanol. The DNA pellet was resuspended in 10 uL distilled water. A T25 flask of QBI-293A cells (Quantum Biotechnologies, Inc. Montreal, Qc. Canada), inoculated the day before and grown to 60-70% confluence, were transfected with the PacI digested DNA. The PacI-digested DNA was diluted up to a total volume of 50 uL with sterile HBS (150 mM NaCl, 20 mM HEPES). In a separate tube, 20 uL DOTAP (Boehringer-Ingelheim, 1 mg/ml) was diluted to a total volume of 100 uL with HBS. The DNA was added to the DOTAP, mixed gently by pipeting up and down, and left at room temperature for 15 minutes. The media was removed from the 293A cells and washed with 5 ml serum-free MEMalpha (Gibco-Invitrogen) containing 1 mM Sodium Pyruvate (Gibco-Invitrogen), 0.1 mM MEM non-essential amino acids (Gibco-Invitrogen) and 25 mM HEPES buffer (Gibco-Invitrogen). 5 mL of serum-free MEM was added to the 293A cells and held at 37° C. The DNA/lipid mixture was added drop-wise to the T25 flask of 293A cells, mixed gently and incubated at 37° C. for 4 hours. After 4 h the media containing the DNA/lipid mixture was aspirated off and replaced with 5 ml complete MEM containing 5% fetal bovine serum. The transfected cells were monitored for GFP expression and plaque formation. Seven days after transfection of 293A cells with the recombinant adenoviral DNA, the cells expressed the GFP protein and began to form visible plaques. The crude viral lysate was collected by using a cell scraper to collect the 293A cells. The lysate was transferred to a 50 mL conical tube. To release most of the virus particles from the cells, three freeze/thaw cycles were done in a dry ice/ethanol bath and a 37° waterbath. The crude lysate was amplified to obtain a working stock of GPHB5 recombinant adenoviral lysate.

B. Amplification and Purification of GPHA2 and GPHB5 Recombinant Adenoviruses.

200 uL of crude recombinant adenoviral lysate was added to each of ten nearly confluent 10 cm plates. The infections were monitored for 48 to 72 hours for cytopathic effect (CPE) under the white light microscope or expression of GFP (GPHB5 virus) under the fluorescent microscope. When all of the 293A cells exhibited CPE, a stock lysate was collected and freeze/thaw cycles performed.

Secondary amplification of the recombinant adenoviruses was achieved with twenty 15-cm tissue culture dishes of 293A cells at 80-90% confluency. Media volume was reduced to 20 mls of 5% MEM and each dish was inoculated with 300-500 uL of amplified stock viral lysate. Complete lysis of the cultures was observed after 48 hours and the lysate collected into 250 ml polypropylene centrifuge bottles. NP-40 detergent was added to a final concentration of 0.5% to ensure complete cell lysis. Bottles were placed on a rotating platform for 10 minutes with rapid agitation. The debris was pelleted by centrifugation at 20,000×G for 15 minutes. The supernatant was transferred to 250-ml polycarbonate centrifuge bottles, and 0.5 volumes of 20% PEG8000/2.5M NaCl solution were added. The bottles were shaken overnight on ice. The bottles were centrifuged at 20,000×G for 15 minutes, and the supernatant discarded. The viral precipitate from 2 bottles was resuspended in 2.5 ml PBS. The resulting virus solution was placed in 2-ml microcentrifuge tubes and centrifuged at 14,000×G for 10 minutes to remove any additional cell debris. The supernatant from the 2-ml microcentrifuge tubes was transferred into a 15-ml polypropylene snap-cap tube and adjusted to a density of 1.34 g/ml with cesium chloride (CsCl). The solution was transferred to 3.2 ml polycarbonate thick-walled centrifuge tubes (Beckman) and spun at (348,000×G) for 3-4 hours at 25° C. The virus formed a white band. Using wide-bore pipette tips, the virus band was collected.

Pharmacia PD-10 columns prepacked with Sephadex G-25M (Pfizer-Pharmacia, New York, N.Y.) were used to desalt the virus preparation. The column was equilibrated with 20 mL of PBS. The virus was loaded and allowed to run into the column. 5 mL of PBS was added to the column and fractions of 8-10 drops collected. The optical densities of 1:50 dilutions of each fraction were determined at 260 nm on a spectrophotometer. A clear absorbance peak was present between fractions 7-12. These fractions were pooled and the optical density (OD) of a 1:10 dilution determined. A formula is used to convert OD into virus concentration: (OD at 260 nm)(10)(1.1×10¹²)=virions/mL. The GPHB5 recombinant adenovirus concentration was 1.99×10¹² virions/mL. The GPHA2 recombinant adenovirus concentration was 6.1×10¹² virions/ml. Glycerol was added to the purified virus to a final concentration of 15%, and stored in aliquots at −80° C.

C. Adenoviral Infection of Mice and Results of Treatment.

Each group consisted of eight female C57BL6 mice. 7.5×10¹¹ particles each of GPHA2- and GPHB5-expressing adenovirus were administered by tail vein injection to the experimental group, while 1.5×10¹¹ particles of adenovirus expressing a parental vector alone were administered to the control group. Animals were sacrificed on day 20 following the injection and tissues were evaluated by a pathologist. Treatment-related effects were observed in the adrenal glands of all eight mice in the experimental group; no effects were observed in the adrenal glands of the control group. The CGH-induced histomorphological changes of the inner adrenal cortical cells included profound hypertrophy and uniformly finely, foamy vacuolization.

D. Intraperitoneal Injection of Recombinant CGH and Results of Treatment.

Sixteen C57BL/6 female mice at 8 weeks of age were separated into four groups. The first group received daily injections of 0.25 mg/kg of recombinant CGH protein intraperitoneally. The second group received daily injections of PBS using the same procedure. The third group received daily injections of 0.25 mg/kg of CGH plus 0.05 mg/kg Dex and the final group received 0.5 mg/kg Dex, alone. Animals were sacrificed on day 15 and the adrenal glands were isolated and weighed. Results are shown in Table 1. Example 5 describes the expression and purification of recombinant CGH protein used in this experiment.

TABLE 1
Significant increase in adrenal weight after chronic CGH treatment.

| | | | Average | |
|---|---|---|---|---|
| | | Number | adrenal | |
| Group | | of | weight/100 g | |
| Number | Treatment | Mice | body weight | P value |
| 1 | 0.25 mg/kg CGH | 4 | 22.26 +/− 0.99 | Group 1 and 2 |
| 2 | PBS | 4 | 15.32 +/− 2.21 | 0.0012 |
| 3 | 0.25 mg/kg CGH + | 4 | 17.66 +/− 1.86 | Group 3 and 4 |
| | 0.5 mg/kg Dex | | | |
| 4 | 0.5 mg/kg Dex | 4 | 13.12 +/− 0.88 | 0.0046 |

### EXAMPLE 5

Expression and Purification of Recombinant CGH

Summary: A Chinese Hamster Ovary (CHO) cell line overexpressing both GPHA2 and GPHB5, the subunits of CGH, was generated and named CHO 180. CHO 180 was found to secrete active, heterodimeric CGH. CGH was purified from the supernatant of CHO 180 using standard biochemical techniques.

A. Generation of CHO 180.

The CGH-producing cell line CHO 180 was generated in two stages. A construct expressing GPHA2, GPHB5 and drug resistance (dihydrofolate reductase) from the CMV promoter was transfected to protein-free CHO DG44 cells (PF CHO) by electroporation. The resulting pool was selected and amplified using methotrexate. Early analysis indicated a high level of GPHA2 expression, but a low level of GPHB5 expression. Therefore, a second construct expressing GPHB5 from the CMV promoter and zeocin resistance from the SV-40 promoter was transfected into the selected, amplified pool by electroporation. After zeocin selection, the final pool (CHO 180) expressed significant levels of both GPHA2 and GPHB5; the proteins were secreted as the non-covalent heterodimer, CGH.

B. Purification of CGH from CHO Culture Supernatant.

CGH was purified from CHO culture supernatant by established chromatographic procedures: first the CGH was captured on a strong cation exchanger, POROS HS50; next it was purified using Hydrophobic Interaction Chromatography with TosoHaas Butyl650S resin; and finally was polished and buffer-exchanged into PBS by Superdex 75 size exclusion chromatography.

C. Cation Exchange Chromatography.

The CHO culture supernatant was 0.2 μm filtered and adjusted to pH 6 and 20 mM 2-Morpholinoethanesulfonic Acid (MES). The CGH in the adjusted supernatant was captured at 55 cm/hr using a 1:2 online dilution with 20 mM MES pH 6 onto a POROS HS 50 column that was previously equilibrated in 20 mM MES pH 6. After loading was complete, the column was washed with 20 column volumes (CV) of equilibration buffer. This was followed by a 3 CV wash with 250 mM NaCl in 20 mM MES pH 6 at 90 cm/hr. Next the CGH was eluted from the column with 3 CV of 500 mM NaCl in 20 mM MES pH 6 at the same flow rate. Finally the column was stripped with steps of 1M and 2M NaCl and then re-equilibrated with 20 mM MES pH 6. The 500 mM NaCl-eluted pool containing the CGH was adjusted at room temperature to 1.0M with (NH₄)₂SO₄ and to pH 6.9 with NaOH for the next step.

D. Butyl 650S Hydrophobic Interaction Chromatography (HIC).

HIC is an adsorptive liquid chromatography technique that separates biomolecules on the basis of net hydrophobicity. The sample is bound to the gel in high salt and then a gradient or step elution of decreasing salt concentration is applied to elute the sample.

The adjusted pool of CGH from the cation exchange chromatography was applied directly at 100 cm/hr to the TosoHaas Butyl650S resin equilibrated in 50 mM NaH₂PO₄ pH 6.9 containing 1.0 M (NH₄)₂SO₄. After loading, the column was washed with 10 CV of equilibration buffer and 10 CV of 50 mM NaH₂PO₄ pH 6.9 containing 0.9M (NH₄)₂SO₄. The CGH was then eluted from the column at 200 cm/hr by reducing the (NH₄)₂SO₄ to 0.5M and collecting 5 CV. This CGH pool was concentrated via ultrafiltration using an Amicon stirred cell with a 5 kDa-cutoff membrane.

E. Size-Exclusion Chromatography.

The concentrated CGH pool was then applied to an appropriately sized bed of Superdex 75 resin (i.e. ≦5% of bed volume) for removal of remaining HMW contaminants and for buffer exchange into PBS. The CGH eluted from the Superdex 75 column at about 0.65 to 0.7 CV and was concentrated for storage at −80 ° C. using the Amicon stirred cell with a 5 kDa-cutoff ultrafiltration membrane. The heterodimeric protein was pure by Coomassie-stained SDS PAGE, had the correct NH2 termini, the correct amino acid composition, and the correct mass by SEC MALS. The overall process recovery estimated by RP HPLC assay was 50-60%.

Additionally, the CGH polypeptide can be expressed in other host systems. The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from. See, e.g., U.S. Pat. No. 4,956,288. Promoters include those from SV-40 or cytomegalovirus, metallothionein genes (U.S. Pat. Nos. 4,579,821 and 4,601,978) and the adenovirus major late promoter. Within an alternative embodiment, adenovirus vectors can be employed. See, for example, Gamier et al., Cytotechnol. 15:145-55, 1994.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

### EXAMPLE 6

The CGH Receptor (TSH-R) is Expressed on Many Different Cells of the Peripheral Immune System.

Whole blood (50 ml) was collected from a healthy human donor and mixed 1:1 with PBS in 50 ml conical tubes. Thirty ml of diluted blood was then underlayed with 15 ml of Ficoll Paque Plus (Pfizer-Pharmacia). These gradients were centrifuged 30 min at 500 g and allowed to stop without braking. The RBC-depleted cells at the interface (PBMC) were collected and washed 3 times with PBS.

Cells were resuspended in FACS Wash Buffer (WB=1× PBS/1% BSA/10 mM Hepes), counted in trypan blue, and 1×10⁶ viable cells of each type were aliquoted into wells of a 96-well round-bottomed plate. Cells were washed and pelleted, then incubated for 20 min on ice with 10 ug/ml of CGH-biotin and cocktails of fluorescently-labeled (FITC and CyChrome) monoclonal antibodies (PharMingen, San Diego, Calif.) recognizing various cell surface markers used to identify particular human immune cell subsets. These markers include the following (listed in the groups of 2 tested in combination with CGH-biotin or a media-only control): CD45RA/CD4, CD56/CD16, CD45RA/CD8, CD14/CD16, CD3/CD19. Cells were washed and then stained with 5 ug/ml streptavidin-PE (PharMingen) for an additional 20 min, to stain CGH-biotin-binding cells. Cells were washed thoroughly and pelleted, then resuspended in 0.4 ml of WB and analyzed on a FACSCalibur using CellQuest software (Becton Dickinson, Mountain View, Calif.).

As shown in TABLE 2, CGH-biotin clearly bound to monocytes, B cells, T cells (both CD4+ and CD8+, not shown), and to NK cells. Additionally, it appeared to bind more avidly to memory phenotype (CD45RA−) CD4+ T cells than to naive (CD45RA+) CD4+ T cells. These data generally agree with the expression pattern of TSH-R determined by RT-PCR (see Example 3) and by immunoprecipitation studies (see Bagriacik and Klein, J Immunol. 164: 6158-65, 2000).

TABLE 2
CGH-biotin binds to a wide variety of immune
cells in human peripheral blood.
Mean Fluorescence Intensities (MFI) are shown for CGH-biotin
(followed by streptavidin-phycoerythrin [SA-PE]) staining of
human PBMC, gated on various immune cell subsets. CGH-biotin
was used at 10 ug/ml. SA-PE was purchased from Pharmingen
and used at 5 μg/ml. These data are representative of 3
independent experiments with different blood donors.

| | | | FL-2 MFI: |
|---|---|---|---|
| | | FL-2 MFI: | CGH-biotin + |
| Cell Subset | Gated On: | 0 + SA-PE | SA-PE |
| Monocytes | CD14+ | 9.1 | 56.4 |
| B cells | CD19+ | 3.8 | 12.9 |
| T cells | CD3+ | 4.3 | 9.1 |
| NK cells | GD56+ | 3.6 | 11.9 |
| Naive CD4+ T cells | CD4+ CD45RA+ | 3.5 | 4.9 |
| Memory CD4+ T cells | CD4+ CD45RA− | 3.4 | 8.7 |

### EXAMPLE 7

CGH Treatment Alters the Production of Inflammatory Cytokinies in the LPS-Induced Mild Endotoxemia Mouse Model

An in vivo experiment was designed to examine the effect of CGH in a mouse model of LPS-induced mild endotoxemia. This model mimics acute endotoxemia/sepsis by challenging mice with a low, non-lethal dose of bacterial endotoxin (lipopolysaccharide, LPS). Serum is collected at various timepoints (1-8 hours) after intraperitoneal LPS injection and analyzed for altered expression of a wide variety of pro- and anti-inflammatory cytokines and acute phase proteins that mediate the inflammatory response. The model provides a means to assess the potential anti-inflammatory effects of therapeutic candidates during a robust inflammatory response. To initially assess the model, we measured proinflammatory cytokines in a pilot experiment to collect reference data for the model.

In this pilot study, six-month old Balb/c (Charles River Laboratories, Wilmington, Mass.) female mice were injected with 25 μg LPS (Sigma) in sterile PBS intraperitoneally (i.p.). Serum samples were collected at 0, 1, 4, 8, 16, 24, 48 and 72 hours from groups of 8 mice for each time point. Serum samples were assayed for inflammatory cytokine levels. IL-1β, IL-6, TNFα, and IL-10 levels were measured using commercial ELISA kits purchased from Biosource International (Camarillo, Calif.).

TNFα levels peaked to 4000 pg/ml and IL-10 levels were 341 pg/ml at 1 hour post-LPS injection. At 4 hours post LPS injection, IL-6, IL-1□ and IL-10 were 6,100 pg/ml, 299 pg/ml and 229 pg/ml, respectively. These results indicated that pro-inflammatory cytokines were indeed produced in this model. From the inflammatory mediators listed above, two were chosen as biological markers for the LPS model of mild endotoxemia: serum TNF□ levels 1 hour post-LPS and serum IL-6 levels 4 hours post-LPS.

C57B1/6 mice (Charles River Laboratories; 5 mice/group) were treated i.p. with PBS, 0.2 mg/kg CGH in PBS, or 2 mg/kg CGH in PBS 1 hour prior to LPS challenge. The mice were then challenged with 25 ug of LPS i.p. and bled at 1 hour and 4 hours after LPS injection. Serum was analyzed for TNF□ (1 hour) and IL-6 (4 hours) levels by ELISA.

Injection of 2 mg/kg CGH protein 1 hour prior to the LPS injection significantly reduced (by about 60%) the TNFα induction at the 1 hour time point, whereas CGH increased serum IL-6 levels by about 70% at the 4 hour time point (TABLE 3, Expt #1). Statistical significance was determined by an unpaired Student's t-test. Similar trends were observed using the lower dose of CGH (0.2 mg/kg), although the differences were not statistically significant (TABLE 3, p values). These results were consistently obtained in 3 independent experiments (TABLE 3). Thus, CGH can suppress the production of the pro-inflammatory cytokine TNFα, while enhancing expression of IL-6, a cytokine that can have either pro- or anti-inflammatory properties. This likely reflects the ability of CGH to increase cAMP levels in immune cells that express TSH-R, leading to changes in the synthesis and secretion of several inflammatory cytokines (see Example 3, Bagriacik and Klein, J Immunol 164: 6158-65, 2000, and Delgado and Ganea, J Biol Chem 274: 31930-40, 1999).

In another experiment, mice were treated with 2 mg/kg of either zlut1 or zsig51 and demonstrated that neither of these monomers had any effect on serum TNFα or IL-6 levels, indicating that the activity of CGH requires the complete heterodimer (data not shown). To determine if CGH would potentiate the effect of a sub-maximal dose of glucocorticoid, groups of 10 C57B1/6 mice each were treated i.p. with PBS, 0.15 or 1.5 mg/kg Dex, 2 mg/kg CGH, or a combination of CGH and low or high doses of Dex, 1 hour prior to injection of 25 ug LPS i.p. As shown in TABLE 3 (Expt #2), either 2 mg/kg CGH or 1.5 mg/kg Dex treatment alone (1 hour prior to LPS) caused a significant drop in serum TNFα levels at 1 hour, as observed in previous experiments. The effects of CGH administered in conjunction with Dex on inhibition of TNFα production were greater than either dose of Dex alone. In particular, the use of CGH with a low dose of Dex substantially decreased the elevation of serum TNFα compared to the low dose of Dex alone. As before, CGH treatment again enhanced increased serum IL-6 levels at 4 hours; however, serum IL-6 levels decreased significantly when the mice received either Dex alone (1.5 mg/kg) or a combination of CGH and Dex (TABLE 3). Thus, the serum IL-6 levels in Dex+CGH treated mice more closely resembled those in mice treated with Dex alone, rather than those treated with CGH alone, suggesting the activity of the glucocorticoid was dominant over that of CGH in this setting.

TABLE 3
CGH treatment reduces TNFα production, and increases IL-6 levels
in the LPS-induced mild endotoxemia mouse model.

| | | | TNFα, | IL-6, | p-value | p-value |
|---|---|---|---|---|---|---|
| | TREATMENT | | pg/ml | ng/ml | vs. PBS | vs. PBS |
| EXPT # | (1 hr prior to LPS) | n = | 1 hour | 4 hours | TNFα | IL-6 |
| 1 | PBS | 5 | 5687 +/− 2310 | 41.1 +/− 10.0 | — | — |
| | 0.2 mg/kg CGH | 5 | 3916 +/− 1057 | 49.7 +/− 3.1 | 0.1576 | 0.1198 |
| | 2 mg/kg CGH | 5 | 2290 +/− 530 | 71.1 +/− 12.9 | 0.0125 | 0.0037 |
| 2 | PBS | 10 | 3115 +/− 891 | 41.2 +/− 11.9 | — | — |
| | 2 mg/kg CGH | 10 | 2274 +/− 524 | 57.1 +/− 16.2 | 0.0191 | 0.0224 |
| | 0.15 mg/kg Dex | 10 | 1765 +/− 589 | 37.1 +/− 8.8 | 0.0008 | 0.7883 |
| | 1.5 mg/kg Dex | 10 | 264 +/− 138 | 16.0 +/− 4.9 | 3.0 × 10⁻⁸ | 0.000008 |
| | 2 mg/kg CGH + 0.15 mg/kg | 10 | 955 +/− 349 | 40.7 +/− 14.8 | 0.000003.1 × 10⁻⁶ | 0.8607 |
| | Dex | | | | | |
| | 2 mg/kg CGH + 1.5 mg/kg | 10 | 238 +/− 82 | 17.3 +/− 5.6 | 0.00000001 | 0.00002 |
| | Dex | | | | | |

### EXAMPLE 8

Delayed Type Hypersensitivity in CGH-Treated Mice

Delayed Type Hypersensitivity (DTH) is a measure of T cell responses to specific antigen. In this response, mice are immunized with a specific protein in adjuvant (e.g., chicken ovalbumin, OVA) and then later challenged with the same antigen (without adjuvant) in the ear. Increase in ear thickness (measured with calipers) after the challenge is a measure of specific immune response to the antigen. DTH is a form of cell-mediated immunity that occurs in three distinct phases 1) the cognitive phase, in which T cells recognize foreign protein antigens presented on the surface of antigen presenting cells (APCs), 2) the activation/sensitization phase, in which T cells secrete cytokines (especially interferon-gamma; IFN-□) and proliferate, and 3) the effector phase, which includes both inflammation (including infiltration of activated macrophages and neutrophils) and the ultimate resolution of the infection. This reaction is the primary defense mechanism against intracellular bacteria, and can be induced by soluble protein antigens or chemically reactive haptens. A classical DTH response occurs in individuals challenged with purified protein derivative (PPD) from Mycobacterium tuberculosis (TB), when those individuals injected have recovered from primary TB or have been vaccinated against TB. Induration, the hallmark of DTH, is detectable by about 18 hours after injection of antigen and is maximal by 24-48 hours. The lag in the onset of palpable induration is the reason for naming the response “delayed type.” In all species, DTH reactions are critically dependent on the presence of antigen-sensitized CD4+ (and, to a lesser extent, CD8+) T cells, which produce the principal initiating cytokine involved in DTH, IFN-□.

In order to test for anti-inflammatory effects of CGH, a DTH experiment was conducted with four groups of C57B1/6 mice treated with: I) PBS, II) 1.5 mg/kg Dexamethasone (Dex), III) 0.2 mg/kg CGH, and IV) 2 mg/kg CGH. All of these treatments were given intraperitoneally two hours prior to the OVA re-challenge. The mice (8 per group) were first immunized in the back with 100 ug chicken ovalbumin (OVA) emulsified in Ribi in a total volume of 200 ul. Seven days later, the mice were re-challenged intradermally in the left ear with 10 ul PBS (control) or in the right ear with 10 ug OVA in PBS (no adjuvant) in a volume of 10 ul. Ear thickness of all mice was measured before injecting mice in the ear (0 measurement). Ear thickness was measured 24 hours after challenge. The difference in ear thickness between the 0 measurement and the 24 hour measurement is shown in TABLE 4. Control mice in the PBS treatment group developed a strong DTH reaction as shown by increase in the ear thickness at 24 hours post-challenge (TABLE 4, Expt #1). In contrast, mice treated with Dex or CGH had a lesser degree of ear thickness compared to controls. These differences were statistically significant, as determined by Student's t-test (TABLE 4, p values vs. PBS).

TABLE 4
CGH inhibits the Delayed Type Hypersensitivity (DTH) reaction when administered
either at the challenge or at the sensitization phase of the response.

| | | | CHANGE IN EAR | | |
|---|---|---|---|---|---|
| | | | THICKNESS (×10⁻³ inch) | | |
| | | TIME/ROUTE OF | LEFT EAR | RIGHT EAR | p value vs. |
| EXPT # | TREATMENT | TREATMENT | (PBS) | (OVA) | PBS |
| | PBS | Challenge (d7) | 0.64 +/− 0.88 | 5.89 +/− 2.32 | — |
| 1 | 1.5 mg/kg Dex | i.p. | 0.42 +/− 0.52 | 2.62 +/− 1.18 | 0.0020 |
| (n = 8) | 0.2 mg/kg CGH | | 0.17 +/− 0.95 | 3.48 +/− 0.79 | 0.0032 |
| | 2.0 mg/kg CGH | | 0.21 +/− 0.34 | 2.48 +/− 1.05 | 0.0145 |
| | PBS | Challenge (d7) | 0.99 +/− 0.56 | 6.64 +/− 0.80 | — |
| 2 | 1.5 mg/kg Dex | i.p. | 0.23 +/− 0.77 | 2.89 +/− 1.29 | 0.000007 |
| (n = 8) | 0.2 mg/kg CGH | | 0.65 +/− 0.63 | 4.41 +/− 0.95 | 0.0002 |
| | 2.0 mg/kg CGH | | 0.67 +/− 1.05 | 3.92 +/− 1.00 | 0.00006 |
| | PBS | Sensitization (d0-4) | 1.50 +/− 0.53 | 7.78 +/− 1.70 | — |
| 3 | 1.5 mg/kg Dex | i.p. | 0.50 +/− 0.54 | 4.38 +/− 1.34 | 0.0014 |
| (n = 7) | 0.2 mg/kg CGH | | 1.31 +/− 0.42 | 4.06 +/− 0.73 | 0.0004 |
| | 2.0 mg/kg CGH | | 1.11 +/− 0.49 | 4.57 +/− 1.58 | 0.0033 |

A second DTH experiment was performed to confirm these results (TABLE 4, Expt #2). Again, CGH and Dex-treated mice exhibited significantly reduced ear swelling in response to the OVA re-challenge (TABLE 4, Expt #2). In DTH experiment #3, CGH was evaluated for anti-inflammatory effects when administered during the sensitization phase of the reaction (i.e. when T cells are responding to the antigen). Mice (7 per group) were administered PBS, Dex or CGH intraperitoneally once a day from days 0 to 4. The mice were then re-challenged with OVA or PBS on day 7 and ear thickness was measured on day 8. Once again, both Dex and CGH significantly inhibited the DTH reaction (TABLE 4, Expt #3), suggesting that CGH can exert anti-inflammatory effects both early and late in the inflammation process.

Ears from mice in DTH experiment #1 were analyzed by immunohistochemistry to assess which cell types were most affected by CGH treatment. Ears were fixed in Zinc/Tris buffer (2.3 mM calcium acetate/31.6 mM zinc acetate/36.7 mM zinc chloride in 0.1M Tris-HCL buffer, pH 7.4) for 24 hours at room temperature and stained with antibodies specific for CD4, CD8, CD11c, and Gr-1 (neutrophils). Although we did not detect staining of CD4, CD8 or CD11c+ cells, there were some interesting differences in the anti-Gr-1 stained sections. Ears were stained using a TechMate 500 autoimmunostainer (Biotech/Ventana) MIP protocol with some modifications. After drying the slides for 1 hour at 60 ° C., the sections were stained with a rat anti-mouse Gr-1 mAb (clone 7/4, Serotec, isotype rat IgG2a, used at 1.25 ug/ml final dilution), overnight at 4° C. This step was followed, after a wash, by biotinylated rabbit anti-rat IgG secondary antibody (Dako, used at 10 ug/ml in PBS with 2% normal rabbit serum and 2% nonfat dry milk) for 45 min. The sections were then washed and treated with HP Block (1.5% H₂O₂ in 50% methanol) 3 times, 7 min each, followed by 25 min in avidin-biotin complex, 3 times of 4 min each in DAB (Diaminobenzidine), then with Methyl green for 10 min.

From this staining procedure, there was a clear reduction in the number of neutrophils infiltrating the ears of those mice treated with CGH or Dex, compared to the PBS-treated controls. Histomorphometry was performed to obtain the average pixel density of neutrophils per unit length (1 mm) present in the ear samples. The results of these analyses are shown in TABLE 5. Despite a fair amount of variability among each group, there was a significant reduction in neutrophil staining in the Dex and low dose CGH groups, as well as a nearly significant (p=0.0507) reduction in the high dose CGH group (see TABLE 5, p values).

TABLE 5
CGH suppresses neutrophil infiltration in the ears of mice
undergoing the DTH response.
Average pixel density, from 4 fields per ear per mouse (n = 4/group).

| | | Pixel density: Neutrophils | | | |
|---|---|---|---|---|---|
| | TIME OF | LEFT EAR | RIGHT EAR | p value vs. PBS | |
| TREATMENT | TREATMENT | (PBS) | (OVA) | LEFT | RIGHT |
| PBS | Challenge | 293 +/− 378 | 12657 +/− 4431 | — | — |
| 1.5 mg/kg Dex | (day 7) | 749 +/− 686 | 3651 +/− 2779 | 0.2877 | 0.0137 |
| 0.2 mg/kg CGH | i.p. | 544 +/− 1044 | 5605 +/− 3725 | 0.6661 | 0.0507 |
| 2 mg/kg CGH | | 273 +/− 399 | 5535 +/− 5331 | 0.9460 | 0.0856 |

CGH anti-inflammatory effects do not seem to be mediated by an increase in corticosteroids by the adrenal cortex.

Since the receptor for CGH, TSH-R, is expressed by the adrenal glands, there was concern that the anti-inflammatory effects observed might be an indirect effect of increasing endogenous corticosteroid production in CGH-treated mice. One well-established side effect of increasing either exogenous or endogenous corticosteroid levels is substantial atrophy of the thymus, as the developing T cells are induced to undergo apoptosis. Therefore, the thymuses of the CGH and Dex-treated mice in the DTH experiments were analyzed. As shown in TABLE 6, while Dex-treated mice exhibited obvious thymic atrophy, neither the thymus weight nor the overall thymocyte cell counts were significantly affected by CGH treatment. Thymocytes were also analyzed by flow cytometry after staining the cells with fluorescently labeled antibodies to CD4, CD8 and CD3 (PharMingen, San Diego, Calif.), and it was found that the relative proportion of each thymocyte subset (CD4 single positive, CD8 single positive, CD4+CD8+ double positive, and CD4−CD8− double negative) in CGH-treated mice was not significantly different from that of the PBS-treated group. Thus, CGH seems to be mediating its anti-inflammatory effects in a manner distinct from that of exgenous glucocorticoids like Dex. This should prove to be an important benefit, as many of the adverse side effects of glucocorticoid treatment might potentially be avoided with CGH therapy.

TABLE 6
Unlike glucocorticoid treatment, CGH treatment in vivo does not induce thymic atrophy.
Thymuses were collected from mice in DTH expt #3 (in Table 5, above).

| TREATMENT | TIME OF | THYMUS | THYMOCYTE | | |
|---|---|---|---|---|---|
| GROUP | TREATMENT | WEIGHT | COUNT | p value vs. PBS | |
| n = 7 | IN DTH EXPT | (mg) | (×10⁻⁶ cells) | Weight | Counts |
| PBS | Sensitization | 61.9 +/− 12.4 | 140.3 +/− 36.6 | — | — |
| 1.5 mg/kg Dex | (days 0-4) | 30.1 +/− 7.6 | 46.0 +/− 7.4 | 0.000004 | 0.0023 |
| 0.2 mg/kg CGH | i.p. | 60.6 +/− 4.8 | 134.0 +/− 24.0 | 0.8856 | 0.7848 |
| 2.0 mg/kg CGH | | 58.43 +/− 20.0 | 128.0 +/− 63.1 | 0.8001 | 0.7485 |

### EXAMPLE 9

Assessment of Lymphoid Tissues in Mice Treated Chronically with CGH

As described above, glucocorticoid treatment results in reductions in immune cell populations, resulting in increased risk of infection. In order to determine whether long-term treatment of mice with CGH might have a deleterious effect on the immune system, C57B1/6 mice were treated with either 300 ug/kg/day human CGH or with PBS (4 mice/group) for a total of 4 weeks. On the last day of treatment, the spleens, peripheral lymph nodes (pooled inguinal, cervical, axillary, and brachial nodes), and thymuses were collected from each group of mice, and single cell suspensions were prepared. Spleens were crushed between two frosted glass slides, while thymuses and lymph nodes were teased apart with forceps, and the cells released were passed over a Nytex membrane (cell strainer) and pelleted. Cells were resuspended in FACS wash buffer (WB=1× Hank's balanced salt solution/1% BSA/10 mM hepes), counted in trypan blue, and 1×10⁶ viable cells of each type were aliquoted into wells of a 96-well round bottom plate. Cells were washed and pelleted, then incubated for 20 min on ice with cocktails of fluorescently-labeled (FITC, PE, and CyChrome) monoclonal antibodies (PharMingen, San Diego, Calif.) recognizing various cell surface markers used to identify particular immune cell subsets. These markers include the following (listed in the groups of 3 tested in combination). For spleen staining: CD11b/Gr1/B220, CD4/CD44/CD8, DX5/NK1.1/CD3; for lymph node staining: CD62L/CD44/CD4, CD62L/CD44/CD8, and CD11b/Gr1/B220; and for thymus staining: CD4/CD3/CD8. Cells were washed thoroughly and pelleted, then resuspended in 0.4 ml of WB and analyzed on a FACSCalibur using CellQuest software (Becton Dickinson, Mountain View, Calif.). As shown in TABLE 7, there were no significant differences (as determined by Student's t-test) in the number (or percentage; data not shown) of each cell population in the lymphoid tissues from the PBS vs. CGH-treated groups of mice.

TABLE 7
Chronic CGH treatment of normal mice treated with 300 ug/kg/day
of CGH for 4 weeks does not affect the cellular distribution in their lymphoid tissues.

| | | NUMBER OF CELLS IN EACH | | |
|---|---|---|---|---|
| | | SUBPOPULATION (millions) | | |
| | | AVERAGE +/− STD DEVIATION | | |
| | GATED CELL | PBS-TREATED | CGH-TREATED | |
| TISSUE | POPULATION | GROUP | GROUP | p value |
| Spleen | TOTAL | 91.8 +/− 21.5 | 99.0 +/− 20.6 | 0.6464 |
| | CD4+ (T cells) | 18.8 +/− 2.60 | 15.3 +/− 3.39 | 0.1475 |
| | CD8+ (T cells) | 12.8 +/− 2.18 | 10.9 +/− 2.47 | 0.3095 |
| | B220+ (B cells) | 54.9 +/− 16.2 | 59.1 +/− 13.28 | 0.7046 |
| | CD11b + Gr1-low | 0.97 +/− 0.43 | 3.40 +/− 3.43 | 0.2091 |
| | (monocytes) | | | |
| | CD11b + Gr1-high | 2.69 +/− 1.37 | 7.35 +/− 7.41 | 0.2624 |
| | (activated granulocytes) | | | |
| | CD11b − Gr1 + (granulocytes) | 5.10 +/− 1.17 | 6.57 +/− 1.21 | 0.1323 |
| | NK1.1 + DX5 + (NK) | 2.32 +/− 0.69 | 3.68 +/− 1.17 | 0.0912 |
| Peripheral | TOTAL | 11.3 +/− 0.60 | 10.9 +/− 4.05 | 0.8552 |
| Lymph | CD4+ | 3.32 +/− 0.44 | 3.23 +/− 1.71 | 0.9290 |
| Nodes | CD8+ | 2.33 +/− 0.29 | 2.54 +/− 1.65 | 0.8124 |
| | B220+ | 6.50 +/− 0.43 | 6.65 +/− 2.54 | 0.9067 |
| Thymus | TOTAL | 111.0 +/− 18.4 | 93.6 +/− 10.0 | 0.1486 |
| | CD4+CD8+ (DP) | 95.2 +/− 15.7 | 79.9 +/− 8.92 | 0.1443 |
| | CD4−CD8− (DN) | 4.36 +/− 0.77 | 4.06 +/− 0.51 | 0.5350 |
| | CD4+CD8− (CD4 SP) | 7.28 +/− 1.28 | 5.72 +/− 0.45 | 0.0616 |
| | CD4−CD8+ (CD8 SP) | 3.91 +/− 0.75 | 3.65 +/− 0.56 | 0.5864 |

These results suggest that although CGH has potent anti-inflammatory activity in vivo, treatment with CGH does not result in depletion of important immune cell populations in the cellular compartments investigated.

### EXAMPLE 10

Binding of ¹²⁵I-CGH and ¹²⁵I-TSH to BHK Cells Expressing the TSHR

Radiolabeled rhCGH and human pituitary-derived TSH (BiosPacific, Emeryville, Calif.) were prepared with ¹²⁵I-Bolton Hunter Reagent (Amersham Pharmacia Biotech, Buckinghamshire, UK) according to the manufacturer's instructions. Fifty μg of protein was radiolabeled to specific activities of 9000-14,000 cpm/ng with 89-97% of the radioactivity precipitating with 10% TCA. Bioactivity of each preparation of ¹²⁵I-CGH and ¹²⁵I-TSH was measured using BHK cells transfected with a CRE-luciferase reporter construct and the human TSHR. There were no significant differences in the bioactivity of ¹²⁵I-CGH or ¹²⁵I-TSH compared to their unlabeled counterparts. BHK cells expressing murine or human TSHRs were cultured in Dulbecco's Modified Eagle's Medium [DMEM (high glucose)] containing 5% fetal bovine serum, 1% Glutamax, 1% sodium pyruvate, 250 nM methotrexate, and Geneticin ( 1/100). Cells were seeded on day 0 at a density of 4,000 cells/cm² and were used on days 4-5 by which time the cell number had increased to about 1×10⁵ cells/cm². Plates of cells were placed on ice and washed twice with 1.0-2.0 ml of ice-cold PBS. The washes were discarded and each well was incubated with the indicated concentration of ¹²⁵I-CGH or ¹²⁵I-TSH in 0.1 mL/cm² of Medium B [DMEM (no bicarbonate), 0.02M Hepes, pH 7.4, 1.0 mg BSA/ml]. Specific binding was determined in the presence (nonspecific binding) and absence (total binding) of unlabeled rhCGH (25.0 μg/ml) or TSH (10 μg/ml). Binding reactions were terminated by removing the binding medium and the cell monolayers were washed three times with 1.0-2.0 ml of ice-cold PBS containing 1.0 mg/ml BSA and twice with 1.0-2.0 ml of ice cold albumin-free PBS. Cells were solubilized in 1.0 ml of 1.0N NaOH for 10 min at room temperature and cell-associated radioactivity was determined in a gamma counter. The measured radioactivity was normalized to cell number that was determined on plates of cells cultured in parallel. Binding data were analyzed by non-linear regression using Prizm 3.0 (GraphPad Software, San Diego, Calif.).

Both ¹²⁵I-CGH and ¹²⁵I-TSH bound to BHK-TSHR cells in a time-dependent manner and reached an apparent steady state within 2 h at 4° C. Binding of both radiolabeled ligands was saturable, specific, and of high affinity (FIG. 1A-B). Scatchard plots of ¹²⁵I-CGH binding were concave upward, indicating either that ¹²⁵I-CGH bound to at least two sites in these cells or that binding exhibited negative cooperativity. Hill plots of the ¹²⁵I-CGH specific-binding data were linear with a Hill slope of 1.08, however, a finding consistent with multiple binding sites for ¹²⁵I-CGH under these conditions. The ¹²⁵I-CGH specific-binding data were directly fitted to a two-site model by non-linear regression to yield the equilibrium dissociation constant (Kd) and the maximum amount bound (Bmax) to each of the sites at steady state. The Kd and Bmax for the high-affinity site were 1.19±0.68 nM and 139±74 fmol/10⁶ cells or 86,000 high affinity sites per cell, respectively. The low-affinity site exhibited a Kd and Bmax of 2.7±2.2 mM and 1.6±1.4×10⁶ fmol/10⁶ cells, respectively. Scatchard plots for ¹²⁵I-TSH specific binding were linear, suggesting the presence of a single binding site (FIG. 1B, inset). Using a single site model of non-linear regression, the Kd and Bmax for ¹²⁵I-TSH binding to BHK-TSHR cells were estimated at 41 nM and 178 fmol/10⁶ cells or about 107,000 sites per cell. Little or no binding of ¹²⁵I-TSH or ¹²⁵I-CGH was observed, in contrast, to parental BHK cells lacking the TSHR (data not shown). Addition of increasing amounts of unlabeled rhCGH reduced the amount of ¹²⁵I-CGH bound to BHK-TSHR cells (FIG. 1C). In contrast, over an identical concentration range, the addition of neither unlabeled rhTSH (FIG. 1C) nor unlabeled rhGPHA2 nor rhGPHB5 (data not shown) reduced the amount of ¹²⁵I-CGH bound. However, rhCGH was about 10- to 20-times more potent as a competitor than either bovine or human TSH in reducing the binding of ¹²⁵I-TSH to BHK-TSHR cells (FIG. 1D).

### EXAMPLE 11

Chronic Treatment of ob/ob Mice with CGH

Summary: CGH was administered daily for 28 days to obese male ob/ob mice. Mice were also treated with vehicle saline and thyroxine. Data was obtained for food intake, blood glucose, serum insulin, serum lipids, and serum thyroid hormone levels. At sacrifice, animals were examined for changes in liver pathology, and gross histology. As described below, CGH treatment resulted in decreased post-prandial glucose and insulin levels, and serum triglyceride and cholesterol levels were significantly reduced compared to controls. Thyroid hormone levels were not elevated above the vehicle group, and the control administration of thyroxine did not produce the same results as the CGH treatment group. Evaluation of liver histology sections was performed to examine the effect of CGH-mediated lipolysis on liver steatosis. Prominent liver steatosis typically associated with the ob/ob strain employed in these studies was significantly reversed by CGH treatment, with treated animals exhibiting marked reduction in fat deposition in liver hepatocytes. Thyroid hormone treatment did not produce a significant change in the extent of steatosis.

Treatment Protocol

11-week old male ob/ob mice were individually caged and given a standard lab chow (4% fat) with free access to food and water. Animals were assigned to a treatment group (n=7−8, average weight 54.3±0.3 g/group), kept on a 12 hour dark cycle (6 PM to 6 AM), and injected each day between 7 and 9 AM. Chow consumed by each animal was weighed twice weekly. All animals received treatments IP in an injection volume of 0.1 ml. CGH was administered at 250 μg/kg, dissolved in sterile saline. Thyroxine (T4) was administered at 1.5 μg/mouse for 4 days, reduced to 1 μg/mouse for 10 days, and returned to 1.5 μg/mouse for the next 14 days. The vehicle controls received sterile saline. TSH was obtained from Genzyme Pharmaceuticals (Thyrogen®, Catalog number 36778; Genzyme Corporation, Cambridge, Mass.), and T4 obtained from Calbiochem, Inc. (EMD Biosciences, catalog number 61205, San Diego, Calif.) All blood draws were performed by retro-orbital puncture under isoflurane anesthesia.

Food Intake

Food intake did not differ significantly between groups (vehicle 5.9±0.22, CGH 6.3±0.09, and thyroxine 6.1±0.17 grams/day of chow).

Measurement of Serum Thyroxine Levels

After 25 days of treatment as described above, blood was sampled from all treated animals, serum separated, and analyzed for total T4 by a commercially available kit (Biocheck, Burlingame, Calif.). After 25 days of treatment, the vehicle T4 levels were 5.14±0.08 μg/dl. The CGH-treated group had T4 levels of 7.25±1.2 μg/dl, and the thyroxine-treated group had T4 levels of 9.04±0.47 μg/dl. The thyroxine treatment group had levels significantly higher than vehicle controls (p<0.001).

Treatment Effects on Glucose and Insulin Levels

Subject animals were fasted for 4 hours at the beginning of the light cycle, and serum was obtained at treatment day 25 under isoflurane anesthesia. Serum Glucose levels were determined with the Cholestech LDX blood analyzer (Cholestech Corporation, Hayward Calif.), and serum insulin levels by ELISA. Serum glucose levels for vehicle and thyroxine treated groups were 306±22 and 295±23 mg/dl, respectively. Serum glucose levels for the CGH treated animals were significantly lower, 251±10 mg/dl, (p<0.05). Serum insulin levels for the vehicle and thyroxine treated groups were 33.5±1.8 and 25.9±1.7 ng/ml, respectively. Serum insulin levels in the CGH treatment group were significantly decreased to 14.2±3.6 ng/ml, (p<0.001).

Serum Lipid Analysis

Subject animals were fasted for 4 hours at the beginning of the light cycle, and serum was obtained at treatment day 25 under isoflurane anesthesia. Triglyceride and total cholesterol levels were determined with the Cholestech LDX blood analyzer. Serum triglyceride levels for the vehicle controls were 143.7±23 mg/dl. The serum triglycerides in the CGH-treated group were lower at 100±14.7 mg/dl, (p=0.09), and the triglycerides in the thyroxine treated group were higher than the vehicle controls at 198±43 mg/dl, (p=0.26). Total cholesterol levels in the vehicle-treated and thyroxine-treated groups were 198±13 and 194±15 mg/dl, respectively. Total cholesterol average of the CGH treatment group was significantly lower at 104±8.8 mg/dl, (p<0.01).

Liver Steatosis

Liver sections were dissected from all treatment groups described above and mounted in paraffin following fixation with NBS-formalin. Sections were mounted and stained with hematotoxylin and eosin (H&E) for visualization of hepatic structural changes. The extent of liver steatosis was evaluated on a four-point scale, from 0 to 3, with zero displaying no signs of liver steatosis, and a score of 4, represented pronounced macrovesicular and microvesicular steatosis. The averages of the groups (n=4) showed significant differences in the extent of steatosis as judged by the size of the lipid inclusions and the integrity of the hepatocyte structure visible in the sections. Average scores given to the groups were vehicle (4), thyroxine (3), and CGH (1.5). The CGH treated animals exhibit a loss of vacuolarization represented by the accumulation of lipid droplets in the hepatocytes. The CGH treated sections appear to have regained hallmarks of normal hepatocyte architecture in only 28 days of treatment, with areas containing hepatocytes without lipid-laden inclusions in the cytoplasm of the cell.

### EXAMPLE 12

CGH Activation of 3T3 L1 Adipocytes and Human Adipocytes Results in cAMP Production

Summary

Differentiated murine 3T3 L1 adipocytes and primary human adipocytes were used to study signal transduction of CGH. 3T3 L1 fibroblasts were differentiated into adipocytes and the cells were transduced with recombinant adenovirus containing a reporter construct, a firefly luciferase gene under the control of cAMP response element (CRE) enhancer sequences. This assay system detects cAMP-mediated gene induction downstream of activation of Gs-coupled G-protein coupled receptors (GPCR's). Treatment of the differentiated 3T3 L1 cells with isoproterenol, a β-adrenoreceptor agonist, resulted in elevation of cAMP levels and an 80-fold induction of luciferase expression. Treatment of differentiated 3T3 L1 cells with CGH also resulted in elevated cAMP levels and a 27-fold induction of luciferase expression. In a separate experiment, undifferentiated 3T3 L1 fibroblasts were transduced with the recombinant adenovirus. Treatment of the fibroblasts with CGH did not result in an increase in reporter gene induction. In another experiment, human primary adipocytes were also transduced with the recombinant adenovirus containing a reporter construct. Treatment of the human adipocytes with isoproterenol produced a 17-fold induction of luciferase expression. Treatment of the human adipocytes with CGH resulted in a 14-fold induction of the reporter gene. These results demonstrate CGH signaling through a GPCR in murine adipocytes and human adipocytes, and the production of cAMP levels similar to those achieved through β-adrenoreceptor stimulation.

Experimental Procedure

3T3 L1 cells were obtained from the ATCC (CL-173) and cultured in growth medium as follows: the cells were propagated in DMEM high glucose (Life Technologies, cat. #11965-092) containing 10% bovine calf serum (JRH Biosciences, cat. #12133-78P). Cells were cultured at 37° C. in an 8% CO2 humidified incubator. Cells were seeded to collagen-coated 96-well plates (Becton Dickinson, cat. #356407) at a density of 5,000 cells per well. Two days later, differentiation medium was added as follows: DMEM high glucose containing 10% fetal bovine serum (Hyclone, cat. #SH30071), 1 μg/ml insulin, 1 μM dexamethasone, and 0.5 mM 3-isobutyl-methyl xanthine (ICN, cat. #195262). The cells were incubated at 37° C. in 8% CO2 for 4 days and the medium replaced with DMEM high glucose containing 10% fetal bovine serum and 1 μg/ml insulin. The cells were incubated at 37° C. in 8% CO2 for 3 days, then the medium was replaced with DMEM high glucose containing 10% fetal bovine serum. The cells were incubated at 37° C. in 8% CO2 for 3 days, and the medium was replaced with DMEM low glucose (Life Technologies, cat. #12387-015) containing 10% fetal bovine serum. The day before the assay, the cells were rinsed with F12 Ham (Life Technologies, cat. #12396-016) containing 2 mM L-glutamine (Life Technologies, cat. #25030-149), 0.5% bovine albumin fraction V (Life Technologies, cat. #15260-037), 1 mM MEM sodium pyruvate (Life Technologies, cat. #11360-070), and 20 mM HEPES. Cells were transduced with AV KZ55, an adenovirus vector containing KZ55, a CRE-driven luciferase reporter cassette, at 5,000 particles per cell. Following overnight incubation, the cells were rinsed once with assay medium (F12 HAM containing 0.5% bovine albumin fraction V, 2 mM L-glutamine, 1 mM sodium pyruvate, and 20 mM HEPES). 50 μl of assay medium were added to each well followed by 50 μl of 2× concentrated test protein. The plate was incubated at 37° C. at 5% CO2 for 4 hours. Medium was removed from the plate and the cells were lysed with 25 μl per well of 1× cell culture lysis reagent supplied in a luciferase assay kit (Promega, cat. #E4530). The cells were incubated at room temperature for 15 minutes. Luciferase activity was measured on a microplate luminometer (PerkinElmer Life Sciences, Inc., model LB 96V2R) following automated injection of 40 μl of luciferase assay substrate into each well. The method described above, with modifications, was also used to test CGH and isoproterenol on human adipocytes obtained from Stratagene (cat. #937236) seeded in 96-well plates. Human adipocytes were rinsed once with basal medium (Stratagene, cat. #220002) containing 0.5% bovine albumin fraction V, then transduced with AV KZ55 at 5,000 particles per cell. Following overnight incubation, the cells were rinsed once with assay medium comprised of basal medium containing 0.5% bovine albumin fraction V and assayed as described above.

### EXAMPLE 13

CGH-Induced Lipolysis in 3T3 L1 Adipocytes

Summary

3T3 L1 Adipocytes were treated with CGH and the non-specific β-adrenoreceptor agonist isoproterenol for 4 hours. Lipolysis was assessed by the accumulation of glycerol and FFAs in the conditioned medium. Measurement of free fatty acids in conditioned media from differentiated 3T3 L1 Cells

Free fatty acids were measured using the Wako NEFA C kit for quantitative determination of non-esterified (or free) fatty acids with a modified protocol. Isoproterenol (ICN), a lipolysis-inducing positive control, was diluted to a starting concentration of 2 μM in assay medium (Life Technologies low glucose DMEM, 1 mM sodium pyruvate, 2 mM L-glutamine, 20 mM HEPES, and 0.5% BSA). The isoproterenol was further diluted in half log serial dilutions. CGH was serially diluted down to 0.06 nM. Medium was removed from 3T3 L1 adipocytes in 96-well plates. 50 μl of assay medium were added to each well, followed by 50 μl of CGH or isoproterenol to each well. The plates were incubated for 4 hours at 37 degrees. 40 μl of conditioned medium were collected for glycerol assay analysis, and 40 μl of conditioned medium were collected for free fatty acid analysis. Oleic acid (Sigma) was dissolved in methanol and used as a reference for determining the amount of free fatty acids in the conditioned media. Wako reagents A and B were reconstituted to 4× the recommended concentration. Conditioned media samples were assayed in 96-well plates. 50 μl of Wako reagent A were added to 5 μl of oleic acid standard plus 40 μl of assay medium. 50 μl of Wako reagent A were added to 40 μl of conditioned medium from differentiated 3T3 L1 cells and 5 μl of methanol. The 96-well plates were incubated at 37□ C for 10 minutes. 100 μl of Wako reagent B were added to each well. The 96-well plates were incubated at 37 degrees for 10 minutes. The 96-well plates were then allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

Measurement of Glycerol in Conditioned Media from Differentiated 3T3 L1 Cells

Glycerol was measured in conditioned media using the Sigma Triglyceride (GPO-Trinder) kit with a modified protocol. Isoproterenol was diluted to a starting concentration of 2 μM. The isoproterenol was further diluted in half log serial dilutions. CGH was diluted to starting concentrations of 300 nM in assay medium. CGH was then serially diluted down to 0.06 nM. Medium was removed from 3T3 L1 adipocytes in 96-well plates. 50 μl of assay medium were added to each well, followed by 50 μl of CGH or isoproterenol to each well. The plates were incubated for 4 hours at 37 degrees. 40 μl of conditioned medium were collected for glycerol assay analysis, and 40 μl of conditioned medium were collected for free fatty acid analysis. The glycerol standard was diluted in water to a range from 200 nmols/10 μl to 0.25 nmols/10 μl. Glycerol was used as a reference for determining the amount of glycerol in the conditioned media. Sigma reagent A was reconstituted to the recommended concentration. Conditioned media samples were assayed in 96-well plates. 150 μl of Sigma reagent A were added to 10 μl of glycerol standard plus 40 μl of assay medium. 150 μl of Sigma reagent A were added to 40 μl of conditioned medium from differentiated 3T3 L1 cells plus 10 μl of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 14

Stimulation of Lipolysis by CGH in Vivo

Summary

CGH, the β3-adrenoreceptor agonist CL 316,243 (CL), and saline vehicle were examined for stimulation of lipolysis in mice following an overnight fast. Mice (n=4) were bled immediately before IP injection of CGH (300 μg/kg), CL (1 mg/kg), or vehicle, and then sacrificed 2 hours later. Lipolysis was assessed as the percent change in serum glycerol or FFA over the 2 hour period. The serum glycerol and FFA for the vehicle groups decreased by 7%±9% and 24%±15%, respectively. The serum glycerol for the CGH group increased by 57%±20%; p=0.0254, and the FFA levels increased 25%±5%; p=0.0188. The serum glycerol for the CL group increased 168%±23%; p=0.0004, and the FFA increased 82%±16%; p=0.0029.

Treatment Protocol

C57 BL/6 male mice, age 19 weeks, were grouped to normalize weight (n=4 for each treatment; average group weight=37.8 g±0.4 g). Mice were housed individually for 18 hours prior to treatment, at which time food was withdrawn, with free access to water given. At approximately 8 a.m., the subjects were anesthetized with halothane and blood samples taken by retro-orbital eye bleed. The blood was allowed to clot, and the serum was separated by centrifugation and frozen for later analysis. Test substances were administered by IP injection in a volume of 0.1 ml, and the animals replaced in their cages for 2 hours with free access to water. At 2 hours, the mice were sacrificed and blood drawn by cardiac puncture.

Measurement of Glycerol and FFA in Murine Serum

For measuring free fatty acids in serum, the method previously described for measuring free fatty acids in conditioned media was followed, with the following modifications. Wako reagents A and B were reconstituted to 2× the recommended concentration. 75 μl of Wako reagent A were added to 5 μl of oleic acid standard plus 5 μl of water. 75 μl of Wako reagent A were added to 5 μl of serum plus 5 μl of methanol (to mirror the oleic acid standard conditions). The 96-well plates were incubated at 37° C. for 10 minutes. 150 μl of Wako reagent B were added to each well. The 96-well plates were incubated at 37° C. for 10 minutes. The 96-well plates were allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

For measuring glycerol in serum, the method previously described for measuring glycerol in conditioned media was followed, with the modifications described below.

Sigma reagent A was reconstituted to 0.5× the recommended concentration. 200 μl of Sigma reagent A were added to 10 μl of glycerol standard. 200 μl of Sigma reagent A were added to 5 μl of serum plus 5 μI of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 15

Intranasal Delivery of CGH

Four different routes of administration for mCGH in female C57BL/6 mice were compared, including intraperitoneal, intramuscular, and subcutaneous injections, as well as intranasal administration. All routes of administration resulted in potent increases in serum thyroxine levels at the first time point analyzed (4 h), and these levels continued to rise by the 8 h time point. There were slight, but insignificant differences between 50 and 250 ug/kg mCGH for all routes, suggesting that, at least in this model, the acute response is close to its maximum following a single dose of >50 ug/kg CGH. Interestingly, intranasal administration was found to be as effective in elevating thyroxine levels as were injections, suggesting that this route could be an easier delivery method than injections.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery.

2. The method of claim 1, wherein the delivery is by intranasal inhalation or pulmonary inhalation.

3. The method of claim 1, wherein the first protein consists of the amino acid sequence from residue 24 to residue 129 of SEQ ID NO: 2.

4. The method of claim 1, wherein the second protein consists of the amino acid sequence from residue 25 to 130 of SEQ ID NO: 5.

5. The method of claim 4, wherein the first protein consists of the amino acid sequence from residue 24 to 129 of SEQ ID NO: 2.

6. The method of claim 5, wherein the delivery is by intranasal inhalation or pulmonary inhalation.

7. A method of reducing the amount of a heterodimeric protein delivered, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, wherein the heterodimeric protein is delivered by aerosol inhalation, and wherein a lesser amount of protein is necessary than by oral, intraperitoneal, intramuscular, or subcutaneous delivery.

8. The method of claim 7, wherein the first protein consists of the amino acid sequence from residue 24 to residue 129 of SEQ ID NO: 2.

9. The method of claim 7, wherein the second protein consists of the amino acid sequence from residue 25 to 130 of SEQ ID NO: 5.

10. The method of claim 9, wherein the first protein consists of the amino acid sequence from residue 24 to 129 of SEQ ID NO: 2.

11. The method of claim 10, wherein the delivery is by intranasal inhalation or pulmonary inhalation.

12. A method of reducing inflammation in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein inflammation is reduced.

13. The method of claim 12, wherein the delivery is by intranasal inhalation or pulmonary inhalation.

14. The method of claim 13, wherein the first protein consists of the amino acid sequence from residue 24 to 129 of SEQ ID NO: 2 and the second protein consists of the amino acid sequence from residue 25 to 130 of SEQ ID NO: 5.

15. A method of inducing lypolysis in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein lypolysis is induced.

16. The method of claim 15, wherein the delivery is by intranasal inhalation or pulmonary inhalation.

17. The method of claim 15, wherein the first protein consists of the amino acid sequence from residue 24 to 129 of SEQ ID NO: 2 and the second protein consists of the amino acid sequence from residue 25 to 130 of SEQ ID NO: 5.

18. A method of reducing liver steatosis in a mammal comprising delivering a heterodimeric protein to a mammal, wherein the heterodimeric protein comprises a first protein and a second protein, wherein the first protein comprises the amino acid sequence from residue 24 to residue 129 of SEQ ID NO: 2, the second protein comprises the amino acid sequence from residue 25 to 130 of SEQ ID NO: 5, and wherein the heterodimeric protein is delivered by aerosol delivery, and wherein liver steatosis is reduced.

19. The method of claim 18, wherein the delivery is by intranasal inhalation or pulmonary inhalation.

20. The method of claim 18, wherein the first protein consists of the amino acid sequence from residue 24 to 129 of SEQ ID NO: 2 and the second protein consists of the amino acid sequence from residue 25 to 130 of SEQ ID NO: 5.
